(19) United States
(12) Patent Application Publication
(10) Pub. No.: US 2016/0231323 A1
(43) Pub. Date: Aug. 11, 2016

(54) PROTECTION OF TCR SIGNALING CHAINS IN CANCER PATIENTS AND ENHANCEMENT OF CAR-T CELL THERAPY

(71) Applicant: Batu Biologics, Inc., San Diego, CA (US)

(72) Inventors: Samuel C. Wagner, San Diego, CA (US); Thomas E. Ichim, San Diego, CA (US); Boris Minev, San Diego, CA (US)

(73) Assignee: Batu Biologics, Inc., San Diego, CA (US)

(21) Appl. No.: 15/018,819

(22) Filed: Feb. 8, 2016

Related U.S. Application Data
(60) Provisional application No. 62/112,974, filed on Feb. 6, 2015.

(51) Int. Cl. G01N 33/574; A61K 9/00; A61K 31/198

(52) U.S. Cl. CPC G01N 33/57492; A61K 31/198; A61K 9/0019; A61K 9/0053; G01N 2333/908; G01N 2333/90283; G01N 2333/54; G01N 2333/545; G01N 2333/5412; G01N 2333/5421; G01N 2333/5431; G01N 2333/525; G01N 2333/918; G01N 2333/523; G01N 2333/96491; G01N 2333/75; G01N 2333/96447; G01N 2333/755; G01N 2333/5754; G01N 2410/02; G01N 2333/90241; G01N 2333/96486

(57) ABSTRACT

Disclosed are means of protecting signaling integrity of T cells in cancer patients through reduction of neutrophil and other cellular induced oxidative stress. In one embodiment the FDA approved drug Mucomyst is administered at a concentration of 50-150 mg/kg to increase expression of T cell receptor (TCR)-zeta chain in patients with cancer. In other embodiments enhancement of CAR-T cell therapy is performed through modulation of inflammatory and oxidative stress in a tumor bearing patient.

### CROSS-REFERENCE TO RELATED APPLICATION

This application claims the benefit of U.S. Provisional Application No. 62/112,974 filed on Feb. 6, 2015, the contents of which are incorporated herein by reference in its entirety.

### FIELD OF THE INVENTION

The invention pertains to the field of cancer immunotherapy, more specifically, the invention relates to the field of inhibiting cancer suppression of the immune system. More specifically, the invention pertains to the field of blocking loss of TCR signaling molecules in cancer patients, thereby allowing recuperation of patient immune functions and possibility of tumor immunotherapy in an effective manner.

### BACKGROUND OF THE INVENTION

The aim of cancer research is the development, advancement, and clinical implementation of therapies that not only destroy, inhibit, or block progression of primary tumors, but also inhibit micrometastatic and metastatic progeny of the primary tumor from seeding the patient. Despite extensive research into the disease, effective means of treating the majority of cancers at present are elusive for the medical community. Although limited success is achieved using the current standard therapies: chemotherapy, radiation therapy, and surgery; each therapy has inherent limitations. Chemotherapy and radiation therapy cause extensive damage to normal, healthy tissue such as bone marrow, intestinal cells, and even neuronal cells, despite efforts to target such therapy to abnormal tissue (e.g., tumors). Surgery is in many cases effective in removing masses of cancerous cells; however, it cannot always ensure complete removal of affected tissue nor are all tumors in an anatomical location amenable to surgical removal.

Immunological control of neoplasia is suggested by evidence of longer survival of patients with a variety of cancers who possess a high population of tumor infiltrating lymphocytes [1-3], the fact that immune suppressed patients develop cancer at a much higher frequency in comparison to non-immune suppressed individuals [4, 5], as well as the evidence that in some situations immunotherapy of cancer is effective [6]. While cancer immunotherapy offers the possibility of inducing remission and control of both the primary tumor mass, as well as micrometastasis, several drawbacks exist. The most significant one is that in many situations immunotherapy is either not feasible, or associated with a variety of toxicities. Various types of immunotherapies for cancer have been tried, including: a) systemic cytokine administration; b) gene therapy; c) allogeneic vaccines; d) autologous vaccine; e) heat shock protein vaccines; f) dendritic cell vaccines; g) tumor infiltrating lymphocytes; h) administration of T cells in a lymphodepleted environment; and i) nutritional interventions. Although each of the approaches contains significant advantages and drawbacks, none of them simultaneously meet the criteria of reproducible efficacy, availability to the mass population, or specificity.

The limitations of many immunotherapeutic approaches to cancer is that tumor antigens are either not clearly defined, or in situations where they are defined, the tumor either mutates to lose expression of such antigens, or the antigen-specific vaccine is only applicable to patients with a certain major histocompatibility complex haplotype. The circumvention of this problem has been attempted using autologous vaccines, however in many cases this is an expensive and difficult procedure. Additionally, one of the most significant hurdles in the area of immunotherapy is the lack of ability to “derepress” immunological abnormalities induced by the tumor to the host.

### DETAILED DESCRIPTION OF THE INVENTION

Cancer patients are known to possess elevated levels of oxidative stress as compared to healthy controls. Didziapetriene et al examined 42 patients with newly diagnosed stages I-IV primary ovary cancer were examined. Level of malondialdehyde (MDA), which correlates with oxidative stress, and catalytic activity catalase (CAT), which is an antioxidant were determined spectrophotometrically. Significantly lower CAT (28.2±15.5 vs. 36.1±14.6 nmol/L/min, P=0.019) activity and higher MDA levels (8.7±3.0 vs. 6.7±2.7 nmol/L, P=0.002) were observed in cancer patients compared with healthy volunteers. Both variables were not confirmed as prognostic factors according to Kaplan-Meier survival estimates [7].

In breast cancer, a more detailed study described enhanced oxidative stress in patients and reduction of this oxidative stress associated with tumor removal. Specifically, plasma samples were collected at diagnosis, and the systemic oxidative profile was determined by evaluating the lipid peroxidation, total antioxidant capacity of plasma (TRAP), MDA, protein carbonylation, and hydroperoxides. Nitric oxide, VEGF, and TNF alpha levels were further measured. Enhanced oxidative stress was detected in patients bearing the primary tumors, characterized by high lipid peroxidation, TRAP consumption, high carbonyl content, and elevated VEGF and TNF-α levels. After tumor removal, the systemic oxidative status presented attenuation in lipid peroxidation, MDA, VEGF, and TNF-α. The 5-year recurrence analysis indicated that all patients who relapsed presented high levels of lipid peroxidation measured by chemiluminescence at diagnosis [8]. Other studies in cancer patients demonstrated similar results for breast [9-15], squamous cell carcinoma [16, 17], gastric cancer [18], liver cancer [19, 20], kidney cancer [21], lung cancer [22-24], multiple myeloma [25], head and neck cancer [26, 27], sarcoma [28], prostate cancer [29], leukemia [30].

In one embodiment, preservation of TCR zeta chain is achieved by decreasing chronic oxidative stress in a cancer patient is decreased by administration of ozone gas in a manner sufficient to stimulate antioxidant enzymes in a patient in need of treatment [31]. Specific concentrations of ozone gas and means of delivery are known in the art. Methodologies of administration, concentrations, and protocols are described in studies of ozone therapy in animals [32], and people [33-37], and are incorporated herein by reference. Methodologies and concentrations of ozone administration vary by disease indication, examples of ozone therapy include treatment of patients with HIV [38], limb ischemia [39-43], heart disease [44, 45], herniated disc [46-50], reduction of tumor hypoxia [51], liver failure [52].

In another embodiment ozone is administered in combination with Q10 enzyme as described [49], to reduce oxidative stress.

In one embodiment the invention provides means to allow for effective induction of tumor immunity through vaccination by induction of re-expression of TCR zeta chain. Immunization may be performed against known tumor antigens to which humoral and cellular based immune responses are known, which include epidermal growth factor receptors (HER2), carcinoembryonic antigen (CEA), mucin (MUC1), the tumor suppressor protein p53, and telomerase reverse transcriptase (TERT) [53-62]. It is known that tumor specific antigens generally initiate an immune response by activation of the sensory component of the immune system. Specifically dendritic cells are known to infiltrate tumors and are associated with increased immunity to tumors [63-66]. T cells are known to stimulate dendritic cell activity.

Accordingly in one embodiment of the invention, protection of TCR zeta chain integrity by reduction of oxidative stress is utilized to increase dendritic cell infiltration of tumors, and thus in turn stimulate antitumor immunity. The importance of preserving dendritic cell activity in the context of the invention is associated with the key importance of the dendritic cell in orchestration of immune response. DC are tissue-fixed cells that are interspersed throughout the body. When DC are activated through contacting an antigen, they migrated to the local lymph node where they activate the synthesis part of the immune system, i.e., the T cells [67]. Evidence of the importance of dendritic cells in the initiation of an immune response came from experiments that demonstrated a vital need for these cells in the generation of a variety of immune responses including in vitro anti-sheep red blood cell responses [68], mixed lymphocyte reaction [69], and in vitro anti□TNP T cell responses [70]. These methodologies are presented to one of skill in the art in the practice of the embodiment of the invention related to restoration of TCR zeta chain signaling as a means of maturing DC in vivo in a cancer patient. In one embodiment of the invention a cancer bearing patient is administered N-acetylcysteine at a concentration of 75 mg/kg intravenously three times per week. As compared to pretreatment, patients treated according to the invention have an increase in the ability of the dendritic cells to stimulate in vitro antibody response, in vitro T cell responses against TNP, and mixed lymphocyte reaction.

One particular embodiment of the invention involves the administration of an anti-oxidant capable of reversing immune suppression seen in many cancer patients. Immune suppression by cancer has been well-documented in advanced cancer patients possessing a variety of malignancies [71-78]. Correlation between immune suppression and poor prognosis has been extensively noted [79-81]. Several means of tumor suppression of immune response are known. For example, a variety of tumor cells possess the ability to induce cleavage of the T cell receptor zeta (TCR-ζ) chain through a caspase-3 dependent manner [82, 83]. Since TCR-ζ is critical for signal transduction, host T cells become unable to respond to tumor antigens. Originally, the TCR-ζ cleavage was described in tumor bearing mice [84, 85] and subsequently in patients [86-91]. The correlation between suppressed TCR-ζ and suppressed IFN-□ production has been reported, implying functional consequences [87]. The cause of TCR-ζ suppression has been attributed, at least in part, to reactive oxygen radicals produced by:
A) The inflammatory activity occurring inside the tumor (it is well established that there is a constant area of necrosis intratumorally;
B) Macrophages associated with the tumor; and
C) Neutrophils activated directly by the tumor, or by the tumor associated macrophages.

Tumors are usually associated with macrophage infiltration, this is correlated with tumor stage and is believed to contribute to tumor progression by stimulation of angiogenesis [92-94].

Cytokines such as M-CSF [92] and VEGF [95] produced by tumor infiltrating macrophages are essential for tumor progression to malignancy. In fact, tumors implanted into M-CSF deficient op/op mice (that lack macrophages) do not metastasize or become vascularized [96]. Tumor-associated macrophages possess an activated phenotype and release various inflammatory mediators such as cyclo-oxygenase metabolites [97, 98], TNF-□ [99], and IL-6 [100] which lead to increased levels of oxidative stress produced by host immune cells. In addition, tumor associated macrophages themselves produce large amounts of free radicals such as NO, OH, and H₂O₂ [101-103]. The high levels of macrophage activation in cancer patients is illustrated by high serum levels of neopterin, a tryptophan metabolite that is associated with poor prognosis [104]. In addition to oxidative stress elaborated by tumor associated macrophages, the presence of the tumor itself causes systemic changes associated with chronic inflammation. Erythrocyte sedimentation ration, C-reactive protein and IL-6 are markers of inflammatory stress used to designate progression of pathological immune diseases such as arthritis [105, 106]. Interestingly advanced cancer patients possess all of these inflammatory markers [107-111]. Another marker of chronic inflammation is decreased albumin synthesis by the liver, this is also seen in cancer patients and is believed to contribute, at least in part, to cachexia [112, 113]. In addition, the inflammatory marker fibrinogen D-dimers is also higher in cancer patients as opposed to controls [114-116]. Schmielau et al reported that in patients with a variety of cancers, activated neutrophils are circulating in large numbers [86]. These neutrophils secrete reactive oxygen radicals such as hydrogen peroxide, which trigger suppression of TCR-ζ and IFN-□ production. This was demonstrated by co-incubation of the neutrophils from cancer patients with lymphocytes from healthy volunteer. A profound suppression of TCR-ζ expression was seen.

Evidence for the critical role of hydrogen peroxide was shown by the fact that addition of catalase suppressed TCR-ζ downregulation. A simple method of assessing the number of circulating activated neutrophils was described in the same paper. This method involves collecting peripheral blood from patients, spinning the blood on a density gradient such as Ficoll, and collecting the lymphocyte fraction. While in healthy volunteers the lymphocyte fraction contained primarily lymphocytes, in cancer patients the lymphocyte fraction contained both lymphocytes and a large number of neutrophils. The reason why these neutrophils are present in the lymphocyte fraction is because activation alters their density so that they co-purify differently on the gradient. A potential indication of the importance of activated neutrophils to cancer progression is provided by Tabuchi et al who show that removal of granulocytes from the peripheral blood of cancer patients resulted in reduced tumor size, unfortunately, the study was performed in only 2 patients [117].

As a mechanism to compensate for immune over-activation, mediators of inflammation have immune suppressive properties. This is best illustrated in the immune suppression seen following immune hyperactivation such as in septic shock. Following the primary scepticemia, patients are systemically immune compromised due to circulating immune suppressive factors that are released in response to the inflammatory stress. This suppression is termed compensatory anti-inflammatory response syndrome (CARS) and is associated with many opportunistic infections and deactivation [118]. The clinical importance of CARS immune suppression is seen in that sepsis survivors show normal T-cell proliferation and IL-2 release, whereas those that succumb possess suppressed T cell responses [119]. Interestingly immune suppressive mediators associated with CARS such as PGE2, TGF-□, and IL-10 are also associated with cancer-induced immune suppression [120].

The role of oxidative stress in sepsis-induced immune suppression was recently demonstrated in experiments where administration of antioxidants (ascorbic acid or n-acetylcysteine) to animals undergoing experimental sepsis blocked immune suppression [121]. Another example of the potential for antioxidants to stimulate immune response in an inflammatory condition is in patients with Duke's C and D colorectal cancer who were administered of a daily dose of 750 mg of vitamin E for 2 weeks. This resulted in restoration of IFN-□ and IL-2 production [122]. The problem of uncontrolled inflammation is seen in sepsis. Although as a monotherapy n-acetylcysteine has little clinical effect, therapeutic administration of n-acetylcysteine results in suppression of the constitutively activated neutrophils seen in these patients [123].

Administration of n-acetylcysteine to smokers results in suppression of markers of oxidative stress [124]. Furthermore, oral n-acetylcysteine administration blocks angiogenesis and suppresses growth of Kaposi Sarcoma [125]. Accordingly, one embodiment of the invention involves administration of n-acetylcysteine at a concentration sufficient to decrease the tumor associated suppression of T cell activity. Such a concentration ranges between 1-10 grams per day, preferably 4-6 grams administered intravenously for a period of type sufficient to normalize production of IFN-□ from PBMC of cancer patients upon ex vivo stimulation. One skilled in the art will understand that n-acetylcysteine is just one example of a compound suitable for reversion of oxidative-stress associated immune suppression. Numerous other compounds may be used, for example ascorbic acid [126-128], co-enzyme Q10 in combination with vitamin E and alpha-lipoic acid [129], genistein [130] or resveratrol [131].

It is known in the art that DC express very high levels of MHC class 2 in comparison to other antigen presenting cells, that allows them to present antigen to T cells at a very efficient rate, as well as being capable of stimulating naïve T cells. This antigen presentation step is where the sensory phase of the immune response encounters the synthesis phase. At this phase not only is antigen presented to T cells, but several secondary signals must also be present. These signals may be cytokines, surface molecules or other soluble mediators. The signals given to the T cell from the antigen presenting cell assist the T cell in determining the type of immune response that will ensue. For example, dendritic cells are potent secretors of the signaling cytokine IL 12 [132-134], this cytokine is needed for the initiation of a Th1 immune response. However, in some immune responses the dendritic cells present antigen but do not provide IL 12, this results in anergy, an antigen specific tolerance characterized by the death or inactivation of the T cells specific for that antigen (207).

In one embodiment antioxidants are used to product TCR zeta chain from degradation also possess properties useful for the treatment of cancer. In one embodiment the antioxidant vitamin C is utilized. It has been previously demonstrated that DCs prepared in the presence of GM-CSF and IL-15 produced more IL-12 upon LPS stimulation as compared to control DC. When control or vitamin C-treated DC were loaded with tumor lysate, mice that received the vitamin C treated DC yielded a higher frequency of CD44(high) CD62L(low) CD8(+) effector and effector memory T cells, which showed an increased ex vivo killing effect of the tumor cells as compared to controls [135]. Other rationale for the utilization of vitamin C as an antioxidant in the production of TCR zeta chain in cancer patients comes from studies demonstrating peripheral benefits of intravenously ascorbic acid in this patient population. Specifically, it is known that cancer, especially in advanced stages is associated with a state of chronic inflammation. In patients with various inflammatory diseases antioxidants, especially ascorbic acid, are consumed leaving the patient in a state of deficiency [136-143]. It is well known that numerous inflammatory conditions including gastritis [144], diabetes [144, 145], pancreatitis [146], pneumonia [147], osteoporosis [148], rheumatoid arthritis [149], are all associated with marked reduction in plasma ascorbic acid levels as compared to healthy controls.

Cancer patients are known to exhibit a general state of chronic inflammation which, as stated above, is related to the tumor itself and the interaction of host factors with the tumor. Elevation in the level of classical inflammatory markers such as fibrinogen [150-156], CRP [157-161], erythrocyte sedimentation rate [162], ferritin [163-166], neopterin [167-169], homocysteine [170, 171], IL-6 [162, 172], and free radical stress [173-176] have been well-documented in cancer patients, with numerous studies demonstrating that elevation is associated with poor survival. The possibility that inflammation itself reduces plasma AA was shown by Fain et al. [177], who examined 184 hospitalized patients and observed that 47.3% suffered from hypovitaminosis C as defined as either depletion (i.e., serum AA levels<5 mg/l) or deficiency (i.e., serum AA levels<2 mg/l). Supporting the use of ascorbic acid (AA), patients with an activated acute phase response, as defined by erythrocyte sedimentation rate above 20 mm and an increase in acute phase reactants (CRP>10 mg/l and/or fibrinogen>4 g/l) had lower serum AA levels. Also associated with decreased serum AA levels was reduction in hemoglobin and albumin. A Japanese population study of 778 men and 1404 women, aged 40-69 years, demonstrated a negative correlation between plasma AA content and CRP [178]. In an interventional study, Block et al. examined 396 healthy nonsmokers randomized to receive either 1000 mg/day vitamin C, 800 IU/day vitamin E, or placebo, for 2 months. A statistically significant decrease in plasma CRP levels was found only in the group receiving AA [179].

Mechanistically, ascorbic acid has been demonstrated to reduce tumor expression of HIF-1 alpha, which results in suppressed angiogenic activity [180, 181]. Direct suppression of proliferating tumor endothelial cells by AA has also been demonstrated [182, 183]. One mechanism of modulation of angiogenesis of cancer by AA may be through the reduction of endothelial permeability in tumor blood vessels, which are known to be extremely leak compared to healthy blood vessels [184]. Additionally, ascorbic acid has a direct killing effect on tumors through a paradoxical oxidative mechanism [185, 186].

In one embodiment the invention may be practiced by the use of intravenous ascorbic acid to reduce TCR-zeta chain cleavage. Ascorbic acid may be used together with immunotherapies or with other antioxidants such as N-acetylcysteine. In a preferred embodiment ascorbic acid is administered according to the Riordan protocol as described and incorporated by reference [187]. Specifics of this protocol are as follows: a) Patients are tested for glucose-6-phosphate dehydrogenase deficiency prior to treatment, as this deficiency can cause hemolysis; b) subjects initially receive of 7.5 to 15 g ascorbate infused by slow drip in saline solution. To ensure that patient has adequate renal function, hydration and urinary voiding capacity, baseline lab tests were performed that include a serum chemistry profile and urinalysis; c) Provided these first treatments are well tolerated, patients are given the option to continue with 25 to 50 g infusions up to three times per week. Other protocols are published in the art for administration of intravenous ascorbic acid to cancer patients that are incorporated by reference [188-202].

Since these dendritic cells seem to play such an important role in immune response initiation, the question arises whether the tumour cell can modulate host dendritic cells so that protective responses will not be initiated. An experiment addressing this question was conducted by Steinbrink et al when his group cultured dendritic cells with IL 10 (a T2 cytokine secreted by tumours). In contrast to controls, IL 10 cultured tumours lost the ability to stimulate in vitro T cell responses. These IL 10 “deactivated” dendritic cells actually induced a state of anergy in the T cells specific for the antigen being presented to.

Another example of tumour-released soluble mediators deactivating dendritic cells was demonstrated by Gabrilovich et al who showed that culturing dendritic cell progenitors with vascular endothelial growth factor (VEGF), a tumour secreted angiogenic factor, prevents dendritic cell maturation and inhibits the ability of these cells to activate T cells. Hypothetically deactivation of the dendritic cell's immunoinitiating abilities, as seen in the aforementioned example, could occur to the dendritic cells that are in close proximity to the tumour. These deactivated dendritic cells would then present tumour antigens but simultaneously send inhibitory signals to the T cells that recognize the tumour antigen, thereby diminishing the host's T cell response against the tumour. Such an explanation could account for observations that cancer patients do not always suffer from systemic immunosuppression: the immunosuppression may be restricted only to tumour antigens.

Although a limited study, the clinical importance of dendritic cells can be seen in an experiment by Enk et al where the dendritic cells of melanoma patients who were either responding to treatment and those from patients who were not responding to treatment were compared. Dendritic cells purified from the tumours of responding individuals had a five-fold greater ability than the dendritic cells of non-responding patients to stimulate a mixed lymphocyte reaction. Also, the dendritic cells from non-responding patients possessed less B 7.2 costimulatory molecule than dendritic cells from responding patients. In addition, the cytokines secreted from the responding patients' dendritic cells where of the Th1 family (IL 2, IL 12, IFN, whereas those secreted by the dendritic cells of the non-responding patients were of the Th2 family (IL 10).

The ability of dendritic cells to assist tumour growth was also assessed by Chaux et al. Utilizing a rat colon carcinoma model they demonstrated the existence of tumour associated dendritic like cells. These cells were presumed to be involved in tumour evasion since they possessed protolerogenic characteristics such as a tumour secreted angiogenic factor, lack of costimulatory molecules but expression of MHC class 2. The concept of tolerogenic dendritic cells is supported by findings in the field of transplantation immunology where passenger leukocytes assist in graft acceptance. These passenger leukocytes are donor-derived dendritic cells that present the alloantigen to recipient T cells. However, the presentation takes place so that instead of entering a state of activation, the recipient T cells enter a state of anergy. In some embodiments tumour antigens are pulsed onto dendritic cells either ex vivo or in vivo by means of targeting. Targeting means include immunoliposomes. Examples of some suitable antigens for use with the current invention include mutated intracellular peptide presented on MHC 1 such as mutant Ras [203], and abnormal mucins that are directly expressed on the tumor surface [204, 205].

In one embodiment of the invention macrophage modulation is performed by administration of antioxidants to reduce the level of oxidative stress and to promote re-expression of TCR-zeta chain and reinstall antitumor immunity. A description of the role of macrophages is provided to assist the practitioner of the invention in its execution. Macrophages are a heterogeneous population of phagocytic cells that provide a front line defense mechanism against invading pathogens or transformed cells. They belong to the sensory part of the immune system. However, the macrophages can also play an effector role when specific immunity is activated and B cells start producing antibodies. The macrophage's effector functions are both antibody dependent and antibody independent. The macrophage functions as an antibody independent effector when it becomes activated and by T cell secreted IFN-(, this endows them with cytotoxic activity toward tumour and virally-infected targets. The antibody dependent macrophage cytotoxicity occurs when the macrophage Fc receptor binds to the Fc portion of antibody/antigen complex. This binding makes the macrophage phagocytose the antigen antibody complex (opsonization), or if the antigen is on the surface of another cell, the macrophage can kill it (antibody dependent cellular cytotoxicity).

The two basic types are the exudate and the resident. Also, resident macrophages can be subdivided into a number of subtypes depending on their anatomical location and function. Generally, exudate macrophages are one of the first cells to enter a site of inflammation. Exudate macrophages are derived from blood born monocytes and cannot proliferate. In contrast, resident macrophages proliferate in response to a variety of stimuli within the tissue they reside in. Macrophage recognition of target cells and pathogens occurs through mechanisms that for the most part are unknown. Known mechanisms of macrophage activation include recognition of cells lacking MHC 1 molecules, recognition of bacterially conserved DNA motifs and binding of LPS or LPS LBP complexes to macrophage receptors. From these presumed mechanism, only the first applies to neoplasia since cancer cells generally express low or absent levels of MHC 1, this is partly to escape detection by T cells. T cells recognize target cells by binding foreign or mutated self peptides that are presented on MHC 1 molecules on the surface of the target cell. Therefore if the target cells downregulates expression of MHC 1 then they can escape T cell attack. Subsequently, the macrophage attacks these cells.

T cell activation is generally a late event (effector stage) in the immune response, since it is known macrophages are one of the first cells the begin infiltration of tumors. Macrophages are called to the site of tissue injury, specifically in cancer or growing cancers, by chemoattractants such as complement components and factors released from damaged cells. This initial leakage of material from dead cells is what Matzinger describes as “danger signals.” When these macrophages recognize the concentration gradient of a chemoattractant, they move through diapedesis toward the source of highest concentration. The macrophages that primarily enter the sites of inflammation are exudate macrophages that derive from the blood-borne monocyte. These macrophages phagocytose material on their way up the concentration gradient. Having reached the area of inflammation they begin to secrete factors that recruits in more cells—primarily T helper cells, but also other cells of the synthesis phase of the immune response. Most helper T cells, however, get activated in the draining lymph node for the area of inflammation.

Besides T cells, a very important cell called into the site of tissue damage by macrophage-released mediators is the dendritic cell, which is key in activating T cells present in the lymph node. Components of the clotting system are also involved in the attraction of macrophages to the site of injury. This is because injury naturally activates the clotting cascades through the extrinsic pathway that involves tissue factor that activate prothrominases to cleave prothrombin into thrombin. Thrombin is a serine protease that acts as a chemotactic agent for macrophages, and induces production of molecules with chemotactic ability which then call in other inflammatory cells such as neutrophils.

It is known that the oxidative stress generated by macrophages and neutrophils contributes to loss of TCR-zeta chain. Thus in one embodiment of the invention the reduction of oxidative stress by administration of agents that suppress neutrophil migration in response to intratumoral macrophages is disclosed. The role of the macrophage in the immune response to tumours is very interesting. Macrophages infiltrate tumours but in some cases they assist in tumor growth. Macrophage infiltration of tumours is widely reported in the clinical literature although no consistent correlation between prognosis and infiltration is evident. For example, in breast cancer, macrophage infiltration was believed to be associated with better prognosis according to a review of the 1970s literature by Underwood. However newer studies argue such infiltration suggests a poor prognosis or bears no prognostic weight. Confusion regarding the prognostic significance of macrophage infiltration is seen in other types of cancers that possess such infiltrates, these include soft tissue sarcoma, ovarian cancer, and hepatoma.

One would expect that the different tumour responses induced by macrophages would depend on the type of macrophage infiltrating, and the type of tumour being infiltrated. For example, tumour secreted products, such as IL 10, have the ability to deactivate macrophages and render them nontumourocidal. Tumours are heterogeneous with regards to IL 10 secretion, thus if a macrophage bound on to a tumour not expressing IL 10, the macrophage may kill the tumour. It is important that the macrophage in contact with the tumour is activated since unactivated macrophages that contact tumour can stimulate tumour proliferation. Tumour heterogeneity can also effect the level of macrophage recruitment. Tumours attract macrophages in part because of the extensive cell damage and death that occurs in the microenvironment of the tumour. This tissue damage usually results in macrophage chemotaxis and the activation of clotting cascades. In fact, some tumour cells express procoagulant tissue factor on their surface, this activates clotting and is partly responsible for the fibrin coating encapsulating many tumours. Since the amount of tissue damage induced by the tumor and the level of tumour bound procoagulant activity is heterogenous with respect to the tumour type, stage of tumour development and tumour vascularization, it should not be a surprise that tumours vary in the amount of macrophages they attract. From the tumour's perspective, macrophages should be avoided since they have the potential to eradicate the tumor. Therefore, it would be expected that tumours secrete agents which prevent the infiltration of macrophages. It is therefore surprising that tumours secrete macrophage chemoattractants such has the macrophage chemoattractant protein 1 (MCP 1). Even more surprising is the observation that transfecting tumour cells with large levels of MCP 1 can increase their immunogenicity, allowing them to act as tumour vaccines. Thus, it appears that tumours secrete just enough MCP 1 to attract macrophages, but not enough to endow them with tumouricidal properties. The reason tumours may desire macrophage recruitment, is to use them to serve the purposes of the tumour (i.e., to provide growth factor and angiogenic support).

An in vivo example of how macrophages are used by tumour cells to facilitate this augmentation of tumour growth and metastasis is the tumour challenged osteopetrotic mouse (op/op mouse). These mice have a genetic abnormality that prevents them from secreting the cytokine monocyte colony stimulating factor (M CSF). Since M CSF is a differentiation factor for the formation of macrophages, the op/op mice almost has an absolute absence of macrophages. When op/op mice are challenged with syngeneic Lewis Lung Cancer (LLC), the tumours grow at a reduced rate compared to wild type mice challenged with LLC. To demonstrate that the impaired growth is due to the lack of macrophages and not an intrinsic anticancer effect associated with the op/op phenotype, the authors administered M CSF to op/op mice and then challenged the mice with LLC. The macrophage containing op/op mice now developed tumors that grew at the same rate as the wild type mice. Most interestingly, the growth of LLC in op/op mice was characterized by a low mitotic index (probably meaning that the macrophages secret growth factors that increase cancer proliferation) and by decreased angiogenesis (implying that the macrophage contributes to tumour angiogenesis).

In one embodiment the invention provides means of activating tumoricidal activities of macrophages while suppressing chronic inflammatory activities and angiogenic activities. This may be accomplished according to the invention through administration of various antioxidants such as n-acetylcysteine, but also together with TLR agonists. Macrophage augmentation of tumour growth has been shown to occur by the following mechanisms: inhibiting activation of Th1 cells and subsequent T cytotoxic responses, secretion of tumour stimulatory growth factors, and induction of angiogenesis. The first mechanism involves having the macrophages take on a “suppressor phenotype”, this can be induced in vitro by culturing them with tumour derived immunosuppressants such as IL 10. Macrophages cultured under such conditions become insensitive to a variety of activatory signals, secrete suppressed levels of inflammatory cytokines such has TNF, IL 1, IL 6 and downregulate costimulatory signals necessary for proper T cell activation such has B 7. Suppression of these functions is detrimental for the host because direct killing of transformed cells will not occur, and more importantly, it activates the synthesis phase of the immune response without the necessary activation signals. If the synthesis phase is activated through a suppressed signal then the response will be molded against what the immune system has perceived to be a weaker danger and therefore the response from the effector phase will be smaller and probably ineffective.

IL 10 induced suppression of B 7 is used by a variety of pathogens to improperly activate the synthesis phase of the immune response. Since the interaction of the macrophage (an antigen presenting cell) with a T cell represents the sensory synthesis communication, it is essential that the macrophage provide the T cell with both an immunogenic antigen, and the necessary costimulatory signals to instruct the T cells in terms of the profile of the immune response to launch. Pathogen downregulation of costimulatory molecules such as B 7 on antigen presenting cells occurs so that the T cell will not be activated to induce the response needed for eradication of the antigen. One means of downregulating B-7 is to increase the concentration of IL 10 in the microenvironment surrounding antigen presentation. An example of this phenomenon is seen in murine schistosomiasis induced granulomas, where the parasite egg somehow triggers the macrophages in the granuloma to secrete IL 10. This IL 10, through an autocrine fashion, inhibits expression of the costimulatory molecule B 7. Since B 7 is not expressed, the macrophages in the granuloma will not be able to activate the T cell response properly and will result in an ineffective response or anergy. If antibodies to IL 10 are given to the granuloma cells surrounding the egg, B 7 is expressed and the T cell becomes properly activated.

In one embodiment of the invention macrophages are reprogrammed through the silencing of immune suppressive genes or silencing of enzymes involved in generation of their metabolites. Such enzymes include PGE-2, IDO, and arginase. Gene silencing is well known in the art and may be accomplished by gene editing using techniques such as CRISPER or TALON, or alternatively utilizing siRNA or shRNA methodologies. It is known that macrophages can be immunosuppressory by secreting soluble mediators that inhibit T cell activation. Since macrophages are one of the first immune cells to enter the tumour microenvironment, secretion of immunosuppressory factors by these cells is likely to inhibit the function of more specific effector cells that enter the tumour at later time points such as the T cell. Should infiltrated macrophages interfere with proper T cell activation and effector function, the tumour may have “devised” a very clever way of using cells of the immune system to protect itself from immune-mediated destruction. Prostaglandin E 2 (PGE 2) is an eicosanoid product of arachinonic acid metabolism via the cyclooxygenase pathway. PGE 2 is associated with inflammatory reactions but also possesses immunomodulatory activity. PGE 2 is secreted by a variety of tumours such as liver, lung, breast, and corectal cancer. In addition, tumour associated macrophages also secrete PGE 2. Heterogeneity in PGE 2 expression has been observed in macrophages, this may be attributed to the different needs of inflammatory mediators at different anatomical sites. For example, microglia cells are resident macrophages of the brain, while these cells perform an excellent job of antigen phagocytosis, they secrete little PGE 2 upon stimulation. In contrast, the exudate macrophages that invade inflamed tissue have the capability of secreting large levels of PGE 2, this allows for inflammation to ensue. Macrophages invading tumour tissue are generally of the exudate subtype, since it is these cells that respond to the chemotactic signal produced by the tumour. PGE 2 inhibits T cell immunity primarily by suppressing the ability of T cells to proliferate, this was demonstrated in head and neck cancer (262), melanoma and colon cancer. In addition, PGE 2 has been shown to inhibit the generation of T cytotoxic cells, important effector cells in antitumour immunity. In vivo, PGE 2 is involved in tumour progression since inhibiting PGE 2 pharmacologically by administering indomethacin significantly impedes metastatic progession in several animal models.

The second method in which the macrophage assists tumour growth is through secreting mitogenic substances. It is important to note that substances present at biological doses that are not mitogenic to nontransformed cells may be mitogenic to neoplastic cells due to typical features of transformed cells such as increased receptor number, decreased receptor turnover, and increased signal transduction ability. Physiologically, the macrophage is involved at the initiation, effector, and resolution of inflammatory responses. Macrophages recognizing LPS or other bacterially derived substances become activated, calling in other inflammatory cells and producing inflammatory mediators. Macrophage effector functions during inflammation typically consist of engulfing cellular debris either through phagocytosis or by virtue of receptor mediated endocytosis. At the end of inflammation the macrophages also are active assisting in tissue repair. In order for macrophages to facilitate wound healing certain agents must be secreted that accelerate the proliferation of the cells that were lost, or induce formation of new blood vessels so proliferation of new cells may begin. In cancer both these processes are induced at inappropriate times and as a result the tumour obtains a survival advantage. Growth factors secreted by activated macrophages during normal wound healing include epidermal growth factor (EGF), platelet derived growth factor (PDGF), transforming growth factor (TGF), and fibroblast growth factor (FGF). To determine if macrophage released growth factors are associated with increased tumour growth, researchers are assaying macrophages at tumour sites for growth factor secretion. Early studies in this field were initiated before cloning of growth factors and had to rely on macrophages extracted from the tumour. In 1981 Currie demonstrated that coculturing of syngeneic macrophages with murine fibrosarcoma cells enhanced proliferation in vitro through the secretion of a soluble factor. A decade later, clinic studies demonstrated that tumour associated macrophages in nasal polyps and lung cancer secrete PDGF at doses that are mitogenic in vitro. Future approaches may be aimed at administering cytokines to diminish macrophage secretion of PDGF. One such cytokine is IFN g, which has been shown to suppress secretion of PDGF by activated macrophage. Tumour associated macrophages have also been shown to secrete EGF at mitogenic concentrations in human breast cancer, although the factors inducing EGF were not investigated. FGF secretion by tumour associated macrophages is important in macrophage induced tumour angiogenesis and tumour proliferation. Similarly, TGF $ can be secreted by macrophages at concentrations that are mitogenic.

The third mechanism in which macrophages assist tumour progression is through the induction of angiogenesis. In order for tumours to grow larger then 3 mm cube, new blood vessels must be formed so that it can receive oxygen and nutrients. The process of new blood vessel formation, called angiogenesis, normally occurs in development and wound healing. Angiogenesis is a multistep process that can be initiated through several means. Tumour hypoxia generally induces expression of VEGF, insulin like growth factor, and IL 8, all of which promote the angiogenic process. It is also interesting that all of the aforementioned agents have immunomodulatory properties. VEGF, for example, is able to alter the antigen presenting ability of dendritic cells by restricting their normal differentiation process. Insulin like growth factor increases monocyte production of IL 1, IL 1 being a potent stimulator of the immunosuppressive mediator PGE 2. IL 8 is a monocyte and neutrophil chemoattractant. Although tumour cells generally initiate their own angiogenesis, this process is greatly assisted by macrophages that secrete contributing factors. One example of this is the ability of the macrophage to degrade matrix. In order for angiogenesis to proceed, tumour matrix must be degraded so endothelial cells can arrive and colonize inside the tumour. Macrophages can degrade tumour matrix by activation of plasmin from plasminogen. Plasminogen is the inactive form of the proteolytic enzyme plasmin, which can cleave matrix proteins such as collagen, fibronectin and laminin. Macrophages activate plasmin from plasminogen by forming a catalyst when the macrophage bound urokinase type plasminogen activator receptor binds serum floating or membrane bound urokinase type plasminogen activator. One way tumour cells can induce macrophages to initiate the angiogenic process is by increasing expression of the of urokinase type plasminogen activator on macrophages. Tumours can modulate macrophage levels of this pro-angiogenic protein by secreting TGF beta. In one embodiment of the invention secretion of TGF-beta is reduced from macrophages by treatment of patient with antioxidants. Both in vitro and in vivo TGF beta was shown to possess this tumour promoting property. Many other pathways that the macrophage could use to induce angiogenesis are likely to exist. Evidence for this was previously discussed, with the example of suppressed angiogenesis in mice lacking macrophages, and increased survival with decreased angiogenesis in breast cancer patients lacking tumour associated macrophages.

In another embodiment of the invention antioxidants are used to reduce oxidative stress produced by granulocytes in cancer patients, which have been demonstrated to reduce expression of TCR zeta chain. Granulocytes are a family of inflammatory cells called polymorphonuclear leukocytes (PMN) which includes neutrophils, eosinophils and basophils. This work will only examine neutrophils due to space limitations, although eosinophils have been shown by some groups to be important in some types of antitumour responses. Neutrophils resemble macrophages in that both are able to phagocytose and kill antigens or antigen coated cells. Neutrophils like macrophages are also considered part of the sensory phase of immune response and part of the effector phase. Since neutrophils are called to the site of inflammation immediately after tissue damage, the immunomodulatory environment formed by the invading neutrophils is very important to how the synthesis phase of the immune response will assess the antigenic threat. Once the synthesis phase of the immune response instructs the initiation of the effector phase, antibody secretion will allow the neutrophils to perform opsonization (305) and antibody dependent cellular cytotoxicity.

Neutrophils possess a multilobed nucleus and granulated cytoplasm in which various bioactive compounds are stored. Neutrophils are short-lived cells that circulate for 7 10 hr and then enter the tissue where they live for another 3 days before undergoing apoptosis. The life of neutrophils can be extended by administration of cytokines such as GM CSF, TNF alpha or interaction with activated platelets. Neutrophils are nonrecycling phagocytic cells in that they release an oxidative burst post phagocytosing the antigen. This destroys the target but also the neutrophil. Neutrophil cytotoxicity occurs through generation of several free radicals (ie. O, H2O2, OH) and halide species (ie. hypochlorous acid) both through myeloperoxidase dependent and independent mechanisms. The free radicals and halides are directly cytotoxic to tumour and nontumour targets partly through induction of membrane damage.

In order for neutrophils to bear any significance on tumour growth, a mechanism of neutrophil homing must exist that guides them to the site of neoplasia. Since tumours are often associated with hypoxia, there is often cell death in various parts of the tumour, this is what elicits inflammatory like reactions such as infiltration of immune cells. Generally, neutrophils nonspecifically enter tissue in response to inflammatory stimuli. Tissue damage releases histamine and other vasoactive compounds which “activate” the endothelium. Neutrophils possess L selectin, a molecule that allows them to roll on activated endothelium through interactions with sialyl Lewis X. Rolling neutrophils then are in closer proximity to accept signals from the endothelium such as the chemotactic signal of IL 8. IL 8 signals neutrophils to express beta 2 integrins that bind on with strong affinity to ICAM 1 on activated endothelium. The beta 2 integrin ICAM 1 binding allows the neutrophil to stop its flow in the blood vessel and to begin transendothelial migration. It is not completely understood what mechanisms the neutrophil uses to move through the extracellular matrix (ECM) although the concentration gradient of the chemotactic signal is the guiding mechanism. Neutrophils secrete cytokines that have been shown to influence the development of immune response.

In one embodiment of the invention neutrophils are used as a source of toll like receptor agonists to stimulate innate immunity. Specifically, ex vivo activation of neutrophils may be induced through treatments known to activate neutrophils to produce neutrophil extracellular traps [206-208], these traps may either be concentrated and administered or admixed with immunogen. In one embodiment of the invention stimulation of neutrophil extracellular trap formation is achieved by incubating blood or neutrophils in a hyperosmolar solution [209].

An early study by Lichtenstein et al in 1985 demonstrated the ability of neutrophils to alter the course of antitumour response. By interperitoneally injecting C. parvum bacteria into ovarian cancer bearing mice, a macrophage mediated tumour reduction was achieved. Surprisingly, neutrophilic infiltrates were essential for the accumulation of tumourolytic macrophages. In addition, adoptive transfer of neutrophils from C parvum injected mice into tumour bearing mice was able elicit a monocyte mediated antitumour response. Today it is known that neutrophils are able to activate macrophages through secretion of cytokines such as interferon gamma, thus molecularly validating the results of Lichtenstein. Neutrophils are also able to influence the type of immune response that will arise by giving specific signals to the synthesis phase. IL 12 is a cytokine secreted by antigen presenting cells such as dendritic cells and macrophages which acts as a costimulatory signal, telling the T helper cell what type of immune response is needed. When the dendritic cell does not secrete IL 12 but presents antigen to the T cell, the T cell will orchestrate a different immune response than if IL 12 was present. Specifically, presentation in conjunction with IL 12 shifts naive T cells to secrete interferon gamma and aquire characteristics of Th1 cells. Th1 cells generate an immune response that is geared toward eradication of intracellular pathogens such as listeria. Since listeria resides intracellularly, antibody responses are useless since antibodies cannot enter the cell. Effective immune response against listeria requires activation of the components of the immune system that can “sense” intracellular abnormalities such as NK cells and T cytotoxic cells. The importance of IL 12 in inducing cellular immunity is demonstrated by experiments indicating the most effective method of inducing immunity to listeria is by coinjecting listeria vaccines with IL 12. Neutrophils play an important role in modulating whether an immune response will be effective against intracellular or extracellular antigens by means of the immunomodulatory signals they secrete. In murine candidiasis an intracellular immune response is protective, and as in listeria, initiation of this response is dependent on IL 12. Neutrophils have been shown to be the greatest secretors of IL 12 in these infections and depletion of neutrophils before candida infection will not allow the protective response to occur). Additional studies by the same group demonstrated neutrophil dependent IL 12 protection could be substituted by exogenous injection of the cytokine in absence of neutrophil. Further, mice recovering from candida had elevated IL 12 and suppressed IL 10 neutrophil production compared to neutrophils from mice succumbing to disease which secreted low IL 12 and high IL 10.

Natural killer (NK) cells are large granular lymphocytes, which have the ability to kill tumour and virally infected target cells without prior sensitization. These cells are nonphagocytic and kill through a variety of mechanisms: secretion of cytotoxic compounds found in their membranes such as granzymes, induction of apoptosis in target cells through fas, and making holes in target cells by releasing perforin. NK cells belong to both the sensory phase and the effector phase of the immune response since they can kill targets upon direct recognition or they can kill via ADCC. The surface phenotype of NK cells is CD3, CD56+, and CD16+. NK cells are important in controlling tumour metastasis since mice lacking NK cytotoxicity (beige mice) have an increase in cancer metastasis compared to controls. There is data which suggests beige mice possess increased incidences of spontaneous tumours, this would indicate a role for the NK cell as the “policemen” of the immune system, guarding the body against development of neoplasia. In fact, NK cells can specifically lyse oncogene transfected fibroblast cell lines while sparing the untransfected control. If NK cells are indeed the frontline defence against neoplasia, patients lacking their activity should have increases in cancer incidence. A Japanese study by Kobayashi demonstrated a 200 fold increased risk of developing malignant neoplasia in humans with Chediak Higashi, a disease comparable to the NK deficiency in beige mice. Cancer patients generally possess suppressed NK activity although there is argument as to whether the NK suppression contributes to the causation of cancer or whether it is an effect, since cancer cells secrete soluble factors that are suppressory for NK cells in vitro. Suppression of cancer patient's NK cells is well known, there are ample studies suggesting that this is related in part through TCR zeta chain cleavage. In one embodiment the invention teaches means of restoring zeta chain signaling in NK cells of patients with cancer through administration of antioxidants.

In some types of cancer, NK activity is positively correlated with improved prognosis. This would agree with in vitro studies that show NK cells have some importance in host response to neoplasia. In order to increase antitumour responses, several investigators have tried to increase NK activity in cancer patients by administration of immunomodulatory compounds such as IL 2. In the trials were IL-2 was able to increase NK activity, the effect of the increase on the tumour mass was minimal. Since anticancer immune response requires a cooperation between various facets of immune response, increasing NK activity alone may not address all the immune abnormalities in the cancer patient. It should therefore not be surprising that IL 2 therapy has not lived up to the expectations placed upon it. In one embodiment of the invention TCR zeta chain or zeta chain on NK cells is restored after IL-2 therapy by combination administration of antioxidants together with IL-2. For the purpose of the invention agents that increase NK activity may be utilized together with antioxidants these include 1.) Hormones, such as prolactin, growth hormone, melatonin and insulin like growth factor. 2) Cytokines, including IL 7, IL 12, IL 15 and IL 18. 3) Neuropeptides such as Met5 enkalphin, beta endorphin and substance P. Understanding interactions between these agents may one day lead to discovery of synergies which can be therapeutically efficacious ways of increasing the NK activity of cancer patients.

Although evidence exists for the importance of NK cells in antitumour immunity, it is still unclear the mechanism through which NK cells mediate this function. NK cells possess antitumour cytotoxic ability in in vitro assays, however the ratio of NK to target cells needed to obtain toxicity is usually 10 100, which is unlikely to occur in vivo considering that NK cells are infiltrating a tumour mass, not the tumour cell infiltrating a NK mass. Realistically the NK to target ratio should be at maximum one to one if the NK cell is touching only the cell at the outermost part of the tumour. Taking this into account we must first speculate that NK cells are more efficacious at combating tumour cells in circulation (because of the higher effector-target ratio) and second, there must exist other antitumour mechanisms besides direct cytotoxicity by which they can induce regression of cancer. Such mechanisms include a recently described proteolytic activity were NK cells can disrupt the three dimensional shape of the tumour without actually killing the tumour cells. Such a disruption would allow other immune cells to enter the tumour, or on the negative side, could promote tumour metastasis. One useful utilization of NK cells for the practice of the invention is to leverage their ability to induce the activation of other immune cells. In one embodiment NK cells are generated from cord blood and used allogeneically in conjunction with systemically administered antioxidants.

1. A method of protecting T cell receptor zeta chain from degradation in a cancer patient comprising of:
a) assessing oxidative stress in said cancer patient;
b) administrating an amount of an antioxidant sufficient to reduce said oxidative stress;
c) assessing said oxidative stress after administration of said antioxidant; and
d) adjusting said dose of said antioxidant to maintain a level of oxidative stress equivalent or below that found in a healthy age-matched subject.

2. The method of claim 1, wherein said assessment of oxidative stress is performed by quantification of serum malondialdehyde (MDA).

3. The method of claim 1, wherein said assessment of oxidative stress is performed by quantification of plasma total antioxidant capacity (TAC).

4. The method of claim 1, wherein said assessment of oxidative stress is performed by quantification of erythrocyte antioxidant enzymes.

5. The method of claim 4, wherein said erythrocyte antioxidant enzymes are selected from a group comprising of: superoxide dismutase (SOD), glutathione peroxidase (GPx) and catalase (CAT).

6. The method of claim 1, wherein said assessment of oxidative stress is performed by quantification of an inflammatory marker.

7. The method of claim 6, wherein said inflammatory marker is selected from a group comprising of: C reactive protein (CRP); IL-1; IL-6; IL-8; IL-11; IL-17; IL-21; IL-33; TNF-alpha; lipoprotein-associated phospholipase A2 (LP-PLA2); lipoprotein Lp(a); myeloperoxidase (MPO); macrophage chemotactic protein 1 (MCP-1); oxidized low-density lipoprotein (oxidized LDL), adiponectin, matrix metalloproteases (MMP), such as MMP-9,1,2; CD40; homocysteine; cardiovascular risk factor (CVRF); plasminogen activator inhibitor (PAI-1); prostaglandin (PG); tissue polypeptide antigen (TPA); von Willebrand factor (vWF); platelet aggregation; fibrinogen; Factor VII; Factor VIII; tissue factor; phosphoglucose (PGI1); endothelin; metaloproteinases; Lipoxygenase; and angiotensin.

8. The method of claim 1, wherein said antioxidant is N-acetylcysteine.

9. The method of claim 8, wherein said N-acetylcysteine is administered intravenously.

10. The method of claim 9, wherein said N-acetylcysteine is administered at a concentration ranging from 50-150 mg/kg of patient weight once to seven times per week.

11. The method of claim 10, wherein said N-acetylcysteine is administered a concentration of 75 mg/kg 3 times per week.

12. The method of claim 8, wherein said N-acetylcysteine is administered in the form of a sustained release (SR) formulation, wherein said SR formulation comprises a prodrug, or analog of N-acetylcysteine, or a salt or solvate thereof; wherein the composition, upon oral administration, provides a therapeutically effective plasma concentration of N-acetylcysteine over more than about 2 hours following the administration.

13. The method of claim 12, further comprising an immediate release (IR) component, wherein the IR component comprises a prodrug, or analog of N-acetylcysteine, or a salt or solvate thereof; wherein the composition, upon oral administration, provides a therapeutically effective plasma concentration of N-acetylcysteine over about 30 minutes to about 24 hours following the administration.

14. The method of claim 12 wherein said SR and/or IR component comprises a prodrug or analog of N-acetylcysteine which is less polar than N-acetylcysteine and is absorbable in the lower gastrointestinal tract of a mammal.

15. The method of claim 12, wherein the SR and/or IR component comprises a prodrug of N-acetylcysteine which has a log P of about 4 or higher.

16. The method of claim 12, wherein said SR and/or IR component comprises a prodrug of N-acetylcysteine which contains a hydrophobic moiety.

17. The method of claim 12, wherein said SR and/or IR component comprises a prodrug of N-acetylcysteine selected from the group consisting of an ester prodrug, an amide prodrug, and an anhydride prodrug.

18. The method of claim 12, wherein said analog of N-acetylcysteine is an amino acid analog.

19. The method of claim 12, wherein the analog of N-acetylcysteine is selected from the group consisting of DiNAC; N-acetylcysteine L-lysine; Carbocisteine; glutathione; S-nitroso-N-acetylcysteine; S-nitrosothiol-N-acetylcysteine; S-allyl-cysteine; S-alkyl-cysteine; N-acetyl-5-farnesyl-cysteine; N-acetyl-L-arginine-NAC; N-acetyl-L-lysine-NAC; N-acetyl-L-histidine-NAC; N-acetyl-L-omithine-NAC; thioester of NAC with salicylic acid; 2′4′-difluoro-4-hydroxy-(1,1′-diphenyl)-3-carboxylic derivatives of NAC; S-allymercapto-NAC (ASSNaC); N,N-diacetyl-L-cystine; N—S-diacyl-cysteine; N-acetylcysteine conjugate of phenethyl isothiocyanate (PEITC-NAC); S-carboxylmethyl-L-cysteine; derivatives of reacting a reactive derivative of p-isobutylphenylpropionic acid and NAC; paraisobutyl NAC; esters of N-acetylcysteine; amides of N-acetylcisteine; anhydrides of N-acetylcistein; and a combination thereof.
